(19) United States
(12) Patent Application Publication
(10) Pub. No.: US 2010/0210518 A1
(43) Pub. Date: Aug. 19, 2010

(54) Peg-lipid conjugates for increasing the solubility of drug compounds

(76) Inventors: Brian Charles Keller, Antioch, CA (US); Nian Wu, North Brunswick, NJ (US)

Correspondence Address: Lee Pederson, 712 East Main Street, Sleepy Eye, MN, 56085, US

(21) Appl. No.: 12/657,611

(22) Filed: Jan. 22, 2010

Related U.S. Application Data
(60) Provisional application No. 61/205,840, filed on Jan. 23, 2009.

(51) Int. Cl. A61K 47/14; A61K 47/22; A61K 31/5377; A61K 31/7048; A61K 31/496; A61K 38/13; A61K 31/404; A61P 29/00; A61P 35/00

(52) U.S. Cl. 514/11; 514/777; 514/786; 514/234.5; 514/31; 514/254.07; 514/420

(57) ABSTRACT

Diacyl lipid-polymer conjugates are used to enhance the solubility of lipophilic drugs in aqueous solution. The conjugates comprise a backbone, two lipophilic acyl groups and a hydrophilic polymer.

### PRIORITY

This application claims priority from U.S. provisional patent application No. 61/205,840 entitled “PEG-lipid conjugates for increasing the solubility of drug compounds” filed Jan. 23, 2009.

### FIELD OF THE INVENTION

The present invention relates to compositions and methods for increasing the solubility of pharmaceutical agents for enhanced delivery. More particularly, the present invention relates to diacyl lipid-polymer conjugates for such increased solubility and enhanced delivery.

### BACKGROUND OF THE INVENTION

Delivery of hydrophobic drug compounds to the site of action is an ongoing problem in pharmaceutical science. Among the types of drug delivery vehicles used are cyclodextrans, drug-lipid complexes, liposomes, and solubilizing agents such as Cremophor®. Various PEG-lipid conjugates have been used in these vehicles. However, many useful drugs are still limited by their solubility, pharmacokinetic profiles and bioavailability. It is therefore an object of this invention to describe new compositions and methods for formulating and delivering lipophilic agents.

### BRIEF DESCRIPTION OF THE INVENTION

Diacyl lipid-polymer conjugates are used to enhance the solubility of lipophilic drugs in aqueous solution. The conjugates comprise a backbone, two lipophilic acyl groups and a hydrophilic polymer.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 is a diagram depicting the cross-section of a liposome made of lipid molecules.

FIG. 2 is a diagram depicting a cross-section of a micelle made of lipid molecules.

FIG. 3 is a diagram depicting a cross-section of a structure made of lipid molecules with large tails relative to the head groups.

FIG. 4 is a space-filling diagram of a lipid molecule having a polar head group and nonpolar hydrocarbon chains.

FIG. 5 shows pharmacokinetic profiles of Gefitinib formulations.

### DETAILED DESCRIPTION

Embodiments of the present invention are described herein in the context of diacyl lipid-polymer conjugates for increasing the solubility and enhancing the delivery of pharmaceutical agents. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Those of ordinary skill in the art will realize that the following description of the present invention is illustrative only and not in any way limiting. Other embodiments of the invention will readily suggest themselves to such skilled persons having the benefit of this disclosure.

The present invention comprises pharmaceutical compositions and methods for making and using same, where the composition comprises a lipophilic drug and one or more polymer-lipid conjugates. The conjugates have a backbone, two acyl groups and a polymer chain. They preferably have a melting temperature less than that of the human body, and are more preferably liquid at room temperature. Aqueous solutions of the conjugates with drugs do not comprise liposomes, but instead comprise a variety of other microstructures.

Definition: As used herein, the term lipophilic' means the ability to dissolve in lipids and/or the ability to penetrate, interact with and/or traverse biological membranes.

U.S. Pat. No. 6,610,322 and its relatives teach the formation of spontaneous liposomes by employing diacyl lipid-polyethleneglycol (PEG) conjugates. That patent describes how to select such PEG-lipid conjugates to form liposomes by simply adding the conjugate to an aqueous solution. It now has been discovered that other similar polymer-lipid molecules are useful for solubilizing hydrophobic drugs without the formation of liposomes.

As described in U.S. Pat. No. 6,610,322, which is herein incorporated by reference, PEG-lipid conjugates will spontaneously form liposomes upon mixing with an aqueous solution if certain conditions are met. For diacylglycerol-PEGs (DAG-PEGs), one condition is that the packing parameters of the total lipids (either a single lipid or a combination of lipids) must lie within ranges that allow liposomes to form. Simply put, the lipids should generally be cone shaped, with tapering that allows them to self-assemble into liposomes. If the head group (including PEG) is too large or too small in relation to the hydrophobic portion or the molecule, liposomes will not form.

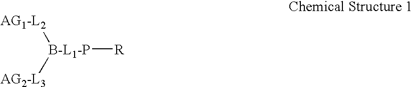

Chemical Structure 1 generally describes the polymer-lipid conjugates of the present invention. AG1 and AG2 indicate acyl groups, i.e., the hydrophobic portion of the molecule. The acyl groups may be the same or different. P indicates a hydrophilic polymer, typically PEG. R indicates the moiety at the distal end of the hydrophilic polymer. B is the backbone. L1 is the linker connecting the backbone to the hydrophilic polymer. L2 and L3 are the linkers connecting the backbone to the acyl groups. The portion of the molecule including B plus L1, L2 and L3 can be considered the extended backbone. The extended backbone plus P plus R comprises the hydrophilic head or headgroup of the molecule. The acyl chains comprise the lipophilic tail.

The amphipathic nature of the lipid-polymer conjugates gives them the ability to increase the solubility of lipophilic drugs in aqueous solution. The acyl groups form associations with the drug, and the head group freely interacts with water. The backbone and linkers may be chosen to confer advantages in solubilizing and/or delivering certain drugs, but the backbone and linkers are generally less functionally important than the hydrophilic polymer and the acyl groups. Thus, a simplified version of the conjugates can be described as in Chemical Structure 2, where EB is the extended backbone.

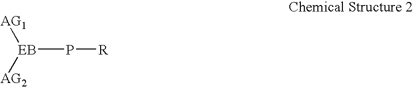

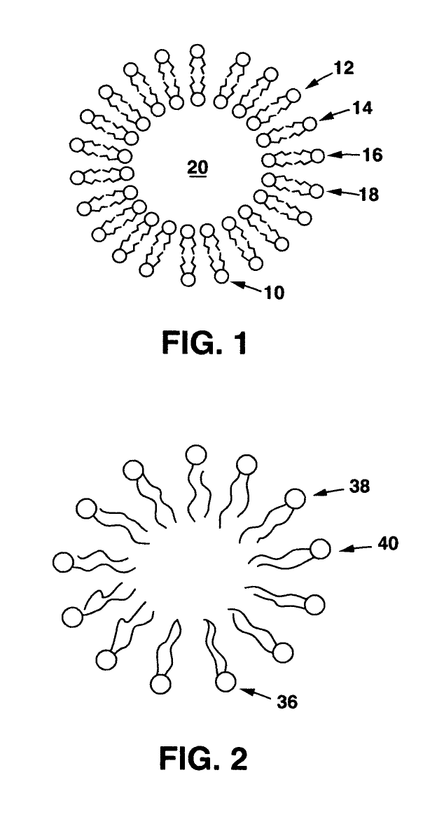

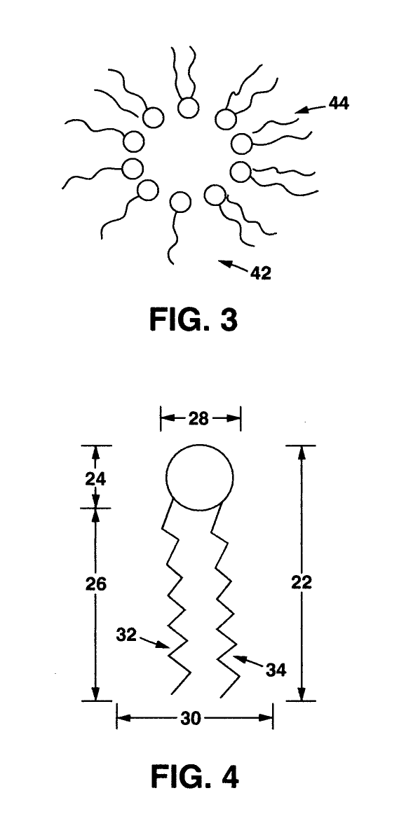

To form a liposome, lipid head groups and hydrocarbon chains must organize themselves into a bilayer with a radius of curvature that allows the bilayer to fold back into itself (see FIG. 1). If the hydrocarbon chains (acyl groups) are too small relative to the head group, the radius of curvature will be too small and micelles will be produced (see FIG. 2). If the hydrocarbon chains are too large relative to the head groups, the radius of curvature will be of the opposite sign and liposomes cannot form (see FIG. 3).

FIG. 1 is a diagram depicting the cross-section of a liposome made of lipid molecules. Liposome 10 comprises a lipid bilayer, made of lipid molecules (e.g., 12, 14, 16, 18), enclosing an aqueous space 20.

FIG. 4 is a space-filling diagram of a lipid molecule having a polar head group and nonpolar hydrocarbon chains. Lipid molecule 22 is comprised of a hydrophilic group 24 and a hydrophobic tail 26. Hydrophobic tail 26 comprises two hydrocarbon chains 32, 34 (AG1 and AG2). While its chemical bonds allow the lipid molecule to be flexible, the head group generally fills an area of diameter 28 while the tail fills an area of diameter 30. Because lipid molecules must be organized in a bilayer to form a liposome, the ratio of the head group diameter to the tail diameter can be neither too large nor too small if liposome formation is to occur.

FIG. 2 is a diagram depicting a cross-section of a micelle made of lipid molecules. Micelle 36 is composed of lipid molecules (e.g., 38, 40). Because the tail groups of the lipid molecules have small diameters relative to the head groups, the lipid molecules organize with a small radius of curvature, and a bilayer cannot form.

FIG. 3 is a diagram depicting a cross-section of a structure made of lipid molecules with large tails relative to the head groups. In FIG. 3, it can be seen that structure 42 forms when lipids (e.g., 44) having large tails relative to the head groups are mixed in aqueous solution. Again, the size ratio between head groups and tails makes bilayer formation impossible.

While FIGS. 1, 2 and 3 have illustrated the basic principle of packing parameters using a single type of lipid molecule, it will be appreciated that the same principle applies to mixtures of lipids. For example, a lipid which has hydrocarbon chains too small to form liposomes as a single species can be mixed with cholesterol to result in a composition which has the proper packing parameters. As another example, a lipid which by itself has the proper packing parameters may form liposomes incorporating limited amounts of other lipids which, by themselves, do not have proper packing parameters. Both single lipids and mixtures of lipids have packing parameters that may be calculated by known methods. In general, liposome compositions which allow liposome formation have packing parameter measurements of Pₐ between about 0.84 and 0.88 and Pᵥ between about 0.88 and 0.93.

Pₐ is the packing parameter with respect to surface and Pᵥ is packing parameter with respect to volume (DD Lasic, Liposomes: From Physics to Applications, Elsevier, pp 51, 1993). The parameters are derived from the equations HCₐ/Tₐ≡Pₐ and HCᵥ/Tᵥ≡Pᵥ where HCₐ is the hydrocarbon chain area, Tₐ is the total area of the molecule, HCᵥ is the volume of the hydrocarbon chains and Tᵥ is the volume of the whole molecule.

Packing parameters can be calculated for mixtures of lipids, since ideal mixing of lipids results in arithmetic average of their individual characteristics. For instance HCₐ/Tₐ≡Pₐ of a binary mixture, in the case of ideal mixing can be expressed as:

<Pₐ>=X₁P₁+X₂P₂,X₁+X₂=1

More generally in the case of i lipids composing a given mixture can be represented by:

<Pₐ>=ΣᵢXᵢPᵢ and ΣᵢXᵢ=1

where Xᵢ is the mole fraction of the lipid in the mixture and Pᵢ is the packing parameter with respect to surface of that lipid.

Two general factors influence the size of the head group in a lipid molecule. One is the actual physical size of the head group. For example, employing a longer PEG chain would make the head group larger. The other is the charge associated with the head group. For example, if the PEG chain was conjugated to the backbone by a phosphodiester bond the phosphate would impart a charge to the head group, effectively increasing its size. In the present invention, non-phospholipids are preferred so that the general means of varying head group size is by varying the length of the polymer, as well as varying the backbone and linkers.

The size of the tail of a lipid is influenced by the length of hydrocarbon chains and degree of saturation in the lipid chain. Single chain lipids will generally not form liposomes, though they may be incorporated into liposomes composed of lipids with two chains. Similarly, lipids with one long chain and one short chain may have relatively small tail sizes.

The presence of sterols and/or drug compounds may also influence liposome formation in a predictable manner. All these above factors must be considered in calculating and predicting whether a single polymer-lipid conjugate will form liposomes, or whether a chemical combination including polymer-lipid conjugates will form liposomes in an aqueous solution. Generally, sterols will not be included in formulations employing the invention.

While liposomes may be a preferred drug delivery vehicle in some cases, it has proven difficult to formulate many lipophilic drugs into liposomal suspensions. Problems have included storage, in vivo stability, drug:lipid ratios, and others. Non-liposomal formulations including the polymer-lipid conjugates of the present invention are useful in many cases, and highly advantageous in particular instances.

Just as it is possible to select PEG-lipid conjugates to from liposomes based on their packing parameters as described in U.S. Pat. No. 6,610,322, it is possible to select pure solutions of polymer-lipid conjugates or mixtures including polymer-lipid conjugates that do not form liposomes. Such selection may be done mathematically, by calculating packing parameters of polymer-lipid conjugates. The selection may be done empirically, by testing various polymer-lipid conjugates. A hybrid approach may also be used, where knowledge of the factors that influence packing parameters is used to predict which conjugates might be useful without rigorously calculating packing parameters before testing for liposome formation.

Table 1 shows a number of lipids which have been tested as single lipids for suitability for the present invention. This table is identical to the table at column 6 in U.S. Pat. No. 6,610,322. Lipids were tested at 2 weight percent in aqueous solution. Note that GDL means glycerol dilaurate, GDO means glycerol dioleate, GDM means glycerol dimyristate, GDP means glycerol dipalmitate, and GDS means glycerol distearate. For each lipid, the number after “PEG” indicates the numbers of C₂H₄O subunits in the PEG chain. The unsaturated dioleate lipids have similar packing parameters to the saturated dimyristate lipids.

TABLE 1

| | melting | | | Spontaneous | Spontaneous | Spontaneous |
|---|---|---|---|---|---|---|
| | point | | | Liposomes at | Liposomes at | Liposomes at |
| Lipid | (° C.) | Pₐ | Pᵥ | 20° C. | 37° C. | 60° C. |
| PEG-23 GDL | Fluid @ 25 | .829 | .869 | NO | NO | NO |
| PEG-12 GDO | Fluid @ 25 | | | YES | YES | YES |
| PEG-23 GDO | Fluid @ 25 | | | NO | NO | NO |
| PEG-45 GDO | 36.3 | | | NO | NO | NO |
| PEG-12 GDM | Fluid @ 25 | .853 | .889 | YES | YES | YES |
| PEG-23 GDM | Fluid @ 25 | .837 | .875 | NO | NO | NO |
| PEG-45 GDM | 33.2 | .823 | .863 | NO | NO | NO |
| PEG-23 GDP | 31.2 | .843 | .880 | | YES | YES |
| PEG-45 GDP | 41.8 | .828 | .867 | NO | NO | NO |
| PEG-12 GDS | 40.0 | .869 | .901 | NO | NO | YES |
| PEG-23 GDS | 39.8 | .849 | .885 | NO | NO | YES |
| PEG-45 GDS | 40.8 | .830 | .870 | NO | NO | NO |

Table 1 demonstrates the liposome forming properties of several polymer-lipid conjugates. To interpret the table, it is necessary to know that spontaneous liposome formation requires that the lipid or lipid mixture be fluid at the temperature of liposome formation. For example, PEG-12 GDM spontaneously forms liposomes at all temperatures tested since it is a liquid at those temperatures and includes PEG in addition to having packing parameters within particular ranges. Similarly, PEG-12 GDO, which shares nearly identical properties to PEG-12 GDM, spontaneously forms liposomes at all temperatures tested. PEG-23 GDS, with a higher melting temperature, only forms liposomes above its melting temperature of 39.8 degrees C.

The polymer-lipid conjugates of the present invention preferably have a melting temperature which allows them to be in liquid form at the temperature of the human body, i.e., below about 37 degrees C. More preferably, the conjugates have melting temperatures below room temperature, i.e., about 25 degrees C. As with packing parameters, melting temperatures may be determined for mixtures of lipids.

Conjugates with lower melting temperatures are preferable for ease of formulation. However, conjugates with higher melting temperatures may be employed by preparing the formulations at higher temperatures.

The GDM series of lipids illustrates the significance of packing parameters. While these lipids all include PEG and are in liquid form at 60 degrees, only PEG-12 GDM demonstrates liposome formation because of favorable packing parameters. The GDS lipid series at 60 degrees illustrates the same point.

Those skilled in the art can practice the present invention by using knowledge of packing parameters to predict and create compositions of polymer-lipid conjugates that will not form liposomes. For example, varying the size of a PEG chain or varying the concentration of PEG-containing lipids in a lipid composition will change packing parameters in a predictable manner. Similarly, varying the backbone and/or linkers also leads to predictable changes to packing parameters. Melting temperatures can also be varied predictably by varying the length of polymer chains, and/or varying the length and saturation of acyl chains.

A wide variety of polymer-lipid conjugates are useful in practicing the present invention as long as several fundamental requirements are met. First, the conjugate must include a hydrophilic polymer. Second, it must include two acyl chains to associate with the lipophilic drug. Third, the packing parameters of the formulation must be such that liposomes will not predominate in aqueous solution. A backbone is also essential, though the acyl chains and hydrophilic polymer tend to dominate the chemical properties of the conjugates in terms of melting temperatures and packing parameters.

When practicing the present invention using aqueous solutions, it is possible that some small fraction of a given formulation may be in the form of liposomes. However, liposomes will at most comprise a minor fraction of the formulations. In many cases, the liposome fraction will be exceedingly small. Generally, a non-liposomal formulation of the present invention has less than 10 percent of the lipid-polymer conjugate in the form of liposomes in dilute aqueous solution. This can also be expressed by saying that liposomes are not the predominant structural form of the conjugate.

Useful conjugates fall into either of two categories: those that do not form liposomes because the head group is too large in relation to the tail, and those that do not form liposomes because the head group is too small in relation to the tail. Because the object of the invention is to increase solubility of lipophilic compounds in aqueous solution, the first category is generally preferred since its compounds have a higher proportion of water soluble elements. However, conjugates with larger lipophilic tails can carry a larger drug load than those with smaller tails. Therefore, preferable acyl groups have at least eight carbons.

Useful acyl groups (AG1 and AG2) include those listed in Table 2 and Table 3.

TABLE 2
Saturated lipids for use in the invention:

| common | | | | Melting |
|---|---|---|---|---|
| name | IUPAC name | Chemical structure | Abbr. | point (° C.) |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 3
Unsaturated lipids

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleic acid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis- | 18:3 |
| | | Δ⁹,Δ¹²,Δ¹⁵ | |
| Arachidonic acid | CH₃(CH₂)₄CH═CHCH₂CH═CHCH₂CH═CHCH₂CH═CH(CH₂)₃COOH^{NIST} | cis,cis,cis,cis- | 20:4 |
| | | Δ⁵Δ⁸,Δ¹¹,Δ¹⁴ | |
| Erucic acid | CH₃(CH₂)₇CH═CH(CH₂)₁₁COOH | cis-Δ¹³ | 22:1 |

While glycerol is the preferred backbone, other useful backbones (B) include 3-carbon chains such as 3-amino-1,2-propanediol; 1-amino-2,3-propanediol; 2-amino-1,3-propanediol; 3-mercapto-1,2-propanediol; 1-mercapto-2,3-propanediol; 2-mercapto-1,3-propanediol; dihydroxybutyric acid; and glyceric acid. Alternatively, sugars may be used as the backbone. Possible sugar backbones include monosaccharides and disaccharides. The backbone may also comprise 4-carbon chains or 2-carbon chains. The backbone may be as simple as N (a single nitrogen atom) or CH, with AG1, AG2 and P bonded directly to the C or N backbone (in which case the linkers can be considered as the shared electron pairs). In addition, amino acids may be used as the backbone. Possible amino acid backbones include cysteine, serine, threonine and asparagine. Cyclic alkanes such as cyclic propane, and heterocyclic compounds such as azoles may also serve as the backbone.

Aromatic compounds such as benzene may also serve as the backbone, although the properties of such aromatic compounds require special consideration. First, aromatic rings are bulky and rigid and may increase the relative size of the headgroup. Second, they are lipophilic in what is typically a hydrophilic portion of the conjugate. Third, stacking between rings may affect the formation of microstructures unpredictably. However, these unique properties may be advantageous in certain situations.

Hydrophilic polymers useful in forming the polymer-lipid conjugates of the invention include polyethyleneglycol (PEG) and other polyalkene oxide polymers, polyoxyethylene alkyl ethers, polyvinylpyrrolidone, Poly(Allyl Amine), Poly(1-glycerol methacrylate), Poly(2-ethyl-2-oxazoline), Poly(2-hydroxyethyl methacrylate/methacrylic acid)/poly(2-hydroxyethyl methacrylate), Poly(2-vinylpyridine), Poly(acrylamide/acrylic acid), Poly(acrylic acid), Poly(butadiene/maleic acid), Poly(ethyl acrylate/acrylic acid), Poly(ethylene oxide-b-propylene oxide), Poly(ethylene/acrylic acid), Poly(methacrylic acid), Poly(maleic acid), Poly(N-iso-propylacrylamide), Poly(N-vinylpyrrolidone/vinyl acetate), Poly(styrenesulfonic acid), Poly(styrenesulfonic acid/maleic acid), Poly(vinyl acetate), Poly(vinyl phosphoric acid), Poly(vinylamine), Polyacrylamide, Polyacrylic Acid, Polyaniline, Polyethylenimine, Pullulan, Polymethacrylamide. Copolymers and block copolymers based on the list above may also be used. The free polymers are water-soluble at room temperature, as well as non-toxic. They do not elicit an appreciable immunogenic response in mammals. Hydrophilic polymers with narrow molecular weight distributions are preferable. Because of already existing acceptance in the pharmaceutical business, PEG is the preferred hydrophilic polymer.

A variety of R groups may be included at the distal end of the polymer. Useful R groups include alkyl groups such as methyl, alkoxy moieties, amines, amino acids, and sugars including monosaccharides, disaccharides, trisaccharides and the oligosaccharides—containing 1, 2, 3, and 4 or more monosaccharide units respectively. Additionally, targeting moieties such as antibody fragments and vitamins may be used as R groups. Generally, the R group is highly soluble in water. The molecular weight of the R group is preferably less than about 200, and more preferably less than about 50. For most applications the R group is preferably not cationic, in order to avoid non-specific binding and decreased circulation time in the bloodstream. However, cationic R groups may be advantageously employed for certain modes of administrations such as topical gels and oral solutions targeting the mouth and throat.

Useful linkers (L1, L2, and L3) include oxy, ester, and carboxyl as well as those shown in table 4.

TABLE 4
Linkers

| No | Symbol | X |
|---|---|---|
| 1 | N₁ | 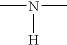 imino |
| 2 | N₂ | —NHNH— |
| | | Hydrazo |
| 3 | N₃ | 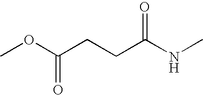 Succinylamino |
| 4 | N₄ | 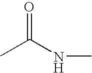 Acetamido |
| 5 | N₅ | 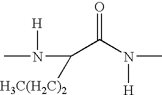 2-aminopentanamido |
| 6 | N₆ | 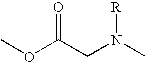 2 (2′)-R′-aminoacetyl R′ = H or alkyl group etc. |
| 7 | N₇ | 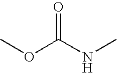 Carbamoyl |
| 8 | N₈ | 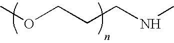 Aminoalkylol or Aminoalkyl, n = 0 to 2 n = 0: amino (N₁) |
| 9 | N₉ | 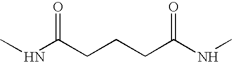 Glutaramido |
| 10 | N₁₀ | 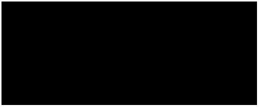 Ornithino |
| 11 | S₁ | —S— |
| | | Thio |
| 12 | S₂ | 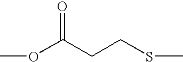 Thiopropanoayl |
| 13 | S₃ | 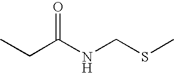 N-(mercaptomethyl)propionamido |
| 14 | S₄ | 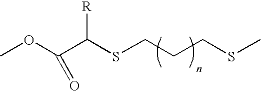 R = H or Alkyl group, n = 0 to 3 R = CH₃ and n = 1: 2-(3-mercaptopropylthio)propanoyl |
| 15 | S₅ | 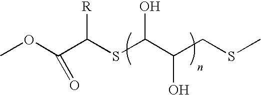 R = H or Alkyl group, n = 0 to 3 R = CH₃ and n = 1: 2-(1,2-dihydroxy-3-mercaptopropylthio)propanoyl |
| 16 | S₆ | 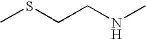 Aminoethanethiol |
| 17 | S₇ | 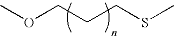 n = 1 to 3 n = 1: mercaptopropanol |
| 18 | S₈ | 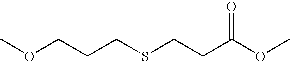 (hydroxypropylthio)propanoayl |
| 19 | O₁ | —O— |
| | | Oxy |
| 20 | Ac₁ | 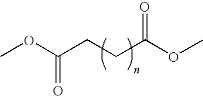 n = 1 to 3. n = 1: Succinyl |
| 21 | Ac₂ | 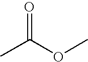 Acetyl |
| 22 | Ac₃ | 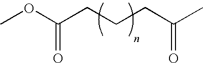 n = 0 to 3. n = 1: oxopentanoyl |

Linkers may be selected to customize the present invention for particular purposes. For example, linkers that are labile at low pH may be preferable in some applications. In other instances, linkers with the ability to form hydrogen bonds with portions of the solubilized drug compound may be advantageous. For most purposes, however, simple linkers such as oxy, thiol, and carboxyl are adequate.

There is a wide range of lipid-polymer conjugates useful in the invention. As mentioned earlier, having PEG as the hydrophilic polymer is preferred. Other preferred embodiments include employing conjugates in preferred molecular weight ranges. When using conjugates with relatively large head groups, it is preferred that the MW of the extended backbone is between about 105 and 250 and that the MW of P plus R is greater, than the combined MW of the acyl groups. Conjugates of the invention have hydrophilic-lipophilic balance ratings between about 8 and about 13.

Some existing solubility enhancing agents such as Labrasol® and Labrafil® are manufactured by conjugating PEG with naturally occurring glyceride mixtures such as apricot pit oil. This results in a solubilizing mixture with a fairly wide range of components. In contrast, the solubility enhancing agents of the present invention are synthesized in discrete steps, resulting in pure well-defined preparations. Having such control allows the formulator to make individual adjustments when using the invention to solubilize a particular lipophilic drug. In some cases, the solubility enhancing agent for a particular lipophilic drug may be a single chemically pure lipid-polymer conjugate of the present invention.

The conjugates of the present invention may be used in conjunction with other solubility enhancing excipients. In preparations of multiple solubility enhancing excipients, the lipid-polymer conjugates of the present invention will preferably comprise at least about 25 percent by weight of all solubility-enhancing excipients, and more preferably at least about 50 percent. When formulated with a lipophilic drug, the lipid-polymer conjugates of the present invention will preferably comprise at least about 20 percent by weight of all solubility enhancing agents plus drug, and more preferably at least about 40 percent. Thus, at low solubility enhancer:lipid ratios the lipid-polymer conjugates of the present invention will comprise a higher percentage of the total solubilizing enhancers. Some illustrative formulations are shown in Table 5. Buffers, water, and excipients that do not enhance solubility (e.g., flavorings) are not included in these calculations.

TABLE 5

| | | | | Lipid-polymer |
|---|---|---|---|---|
| | | Total | | conjugates (% of |
| Amount | Solubility | solubility | Lipid-polymer | total solubility |
| of drug | enhancer:drug | enhancers | conjugates | enhancers plus |
| (mg) | ratio | (mg) | (mg) | drug) |
| 1 | 0.5 | 0.5 | 0.3-0.5 | 20.0-33.3 |
| 1 | 1.0 | 1.0 | 0.4-1.0 | 20.0-50.0 |
| 1 | 2.0 | 2.0 | 0.6-2.0 | 20.0-66.7 |
| 1 | 5.0 | 5.0 | 1.25-5.0 | 20.8-83.3 |
| 1 | 10.0 | 10.0 | 2.5-10.0 | 22.7-90.9 |
| 1 | 20.0 | 20.0 | 5.0-20.0 | 23.8-95.2 |
| 1 | 50.0 | 50.0 | 12.5-50.0 | 24.5-98.0 |

To be clear, the term lipid-polymer conjugate when used herein is meant specifically to only include the conjugates of the present invention. Though existing solubility enhancers may include conjugates of lipids and polymers, they are not included in the term lipid-polymer conjugates for the purposes of this specification.

Unlike naturally occurring lipids such as phospholipids, the conjugates of the present invention do not have a critical micellar concentration (CMC). Micelles only form when the concentration of surfactant is greater than the CMC, and the temperature of the system is greater than the critical micelle temperature. The present polymer-lipid conjugates form aggregates spontaneously at any given concentration.

Formulations of drugs with polymer-lipid conjugates in aqueous solution can be further enhanced by sizing the particles. Conventional technologies such as micronizing, wet-milling or spray-drying can be utilized for particle size reduction. Preferable particle sizes are 0.05 to 25 micron; more preferable particles are 0.05 to 10 micron; Most preferable particle sizes are 0.1 to 5 micron.

The present invention is useful in a variety of situations, and provides advantages over the prior art in several different ways. Problems with lipophilic drug preparation, solubility and delivery can be reduced by employing the invention.

Pharmaceutical preparations using the lipid-polymer conjugates can be used in a variety of ways. At high concentrations in aqueous solutions (greater than about 20% w/w), the preparations are gel-like and suitable for topical administration. Gels can also be formulated as capsules for oral or rectal administration. Preparations of more dilute aqueous solution are useful for oral, intravenous, topical, and subcutaneous administration.

The invention may be used to increased the solubility of any lipophilic drug in any mode of administration. Though certain specific lipophilic drugs are listed in this specification, the invention is intended to be used with all lipophilic drugs. The lipid-polymer conjugates may also be used to formulate cosmetics, nutrients and nutriceuticals, either as gels or as more dilute solutions.

In one aspect the invention is a composition for enhancing the solubility of a lipophilic drug, where the composition comprises at least 25 percent by weight of a diacyl lipid-polymer conjugate. The diacyl lipid-polymer conjugate represented by the formula

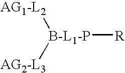

where AG1 and AG2 are acyl groups; where L1, L2 and L3 are linkers; where B is a backbone; where P is polyethyleneglycol; where R is not cationic; where AG1 and AG2 together comprise the lipophilic tail portion of the conjugate; where B, L1, L2, L3, P and R together comprise the hydrophilic head portion of the conjugate; and where the head portion is too large in relation to the tail portion to allow a pure aqueous solution of the conjugate to predominantly form liposomes. The melting temperature of the conjugate is preferably less than about 36 degrees centigrade, and more preferably less than about 25 degrees centigrade. The backbone is preferably glycerol or a sugar.

In another aspect the invention is a pharmaceutical composition comprising a lipophilic active agent and one or more solubility enhancers including a diacyl lipid-polymer conjugate, said diacyl lipid-polymer conjugate represented by the formula

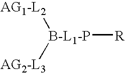

where AG1 and AG2 are acyl groups; where L1, L2 and L3 are linkers; where B is a backbone; where P is polyethyleneglycol; where R is not cationic; where AG1 and AG2 together comprise the lipophilic tail portion of the conjugate; where B, L1, L2, L3, P and R together comprise the hydrophilic head portion of the conjugate; and where the head portion is too large in relation to the tail portion to allow a pure aqueous solution of the conjugate to predominantly form liposomes; and where said conjugate comprises at least about 20 percent of the combined mass of the drug and all solubility enhancers. The melting temperature of the conjugate is preferably less than about 36 degrees centigrade, and more preferably less than about 25 degrees centigrade. The backbone is preferably glycerol or a sugar.

In another aspect the invention is a method of enhancing the solubility of a lipophilic drug comprising combining the drug in an aqueous solution with one or more solubility enhancers including a diacyl lipid-polymer conjugate, said diacyl lipid-polymer conjugate represented by the formula

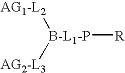

where AG1 and AG2 are acyl groups; where L1, L2 and L3 are linkers; where B is a backbone; where P is polyethyleneglycol; where R is not cationic; where AG1 and AG2 together comprise the lipophilic tail portion of the conjugate; where B, L1, L2, L3, P and R together comprise the hydrophilic head portion of the conjugate; where the head portion is too large in relation to the tail portion to allow a pure aqueous solution of the conjugate to predominantly form liposomes; and where said conjugate comprises at least about 20 percent of the combined mass of the drug and all solubility enhancers. The melting temperature of the conjugate is preferably less than about 36 degrees centigrade, and more preferably less than about 25 degrees centigrade. The backbone is preferably glycerol or a sugar.

In another aspect the invention is a method treating a mammal with a lipophilic drug comprising combining the drug in an aqueous solution with one or more solubility enhancers including a diacyl lipid-polymer conjugate, said diacyl lipid-polymer conjugate represented by the formula

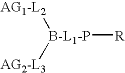

where AG1 and AG2 are acyl groups; where L1, L2 and L3 are linkers; where B is a backbone; where P is polyethyleneglycol; where R is not cationic; where AG1 and AG2 together comprise the lipophilic tail portion of the conjugate; where B, L1, L2, L3, P and R together comprise the hydrophilic head portion of the conjugate; where the head portion is too large in relation to the tail portion to allow a pure aqueous solution of the conjugate to predominantly form liposomes; where said conjugate comprises at least about 20 percent of the combined mass of the drug and all solubility enhancers; and administering the solution to the mammal. The melting temperature of the conjugate is preferably less than about 36 degrees centigrade, and more preferably less than about 25 degrees centigrade. The backbone is preferably glycerol or a sugar.

In another aspect the invention is a composition for enhancing the solubility of a lipophilic drug, said composition comprising at least 25 percent by weight one or more diacyl lipid-polymer conjugates, said diacyl lipid-polymer conjugates represented by the formula

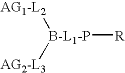

where AG1 and AG2 are acyl groups; where L1, L2 and L3 are linkers; where B is a backbone; where P is a hydrophilic polymer; where R has a molecular weight less than about 200; and where said diacyl lipid-polymer conjugate has packing parameters which do not allow for the formation of liposomes. The melting temperature of the conjugate is preferably less than about 36 degrees centigrade, and more preferably less than about 25 degrees centigrade. In one embodiment, the head group is proportionally too large to allow the formation of liposomes. In another embodiment, the head group is proportionally too small to allow the formation of liposomes. The backbone is preferably glycerol or a sugar. The polymer is preferably PEG.

In another aspect the invention is a pharmaceutical composition comprising an active agent and one or more diacyl lipid-polymer conjugates, said diacyl lipid-polymer conjugate represented by the formula

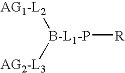

where AG1 and AG2 are acyl groups; where L1, L2 and L3 are linkers; where B is a backbone; where P is a hydrophilic polymer; where R has a molecular weight less than about 200; where said diacyl lipid-polymer conjugate has packing parameters which do not allow for the formation of liposomes; and where said conjugate comprises at least about 20 percent of the composition by mass. The melting temperature of the conjugate is preferably less than about 36 degrees centigrade, and more preferably less than about 25 degrees centigrade. In one embodiment, the head group is proportionally too large to allow the formation of liposomes. In another embodiment, the head group is proportionally too small to allow the formation of liposomes. The backbone is preferably glycerol or a sugar. The polymer is preferably PEG. The composition may further comprise an aqueous solution.

In another aspect the invention is a method of enhancing the solubility of a lipophilic drug comprising combing the drug in an aqueous solution with one or more solubility enhancers including one or more diacyl lipid-polymer conjugates, said diacyl lipid-polymer conjugates represented by the formula

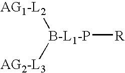

where AG1 and AG2 are acyl groups; where L1, L2 and L3 are linkers; where B is a backbone; where P is a hydrophilic polymer; where R has a molecular weight less than about 200; and where said diacyl lipid-polymer conjugate has packing parameters which do not allow for the formation of liposomes; and where said conjugate comprises at least about 20 percent of the combined mass of the drug and all solubility enhancers. The melting temperature of the conjugate is preferably less than about 36 degrees centigrade, and more preferably less than about 25 degrees centigrade. In one embodiment, the head group is proportionally too large to allow the formation of liposomes. In another embodiment, the head group is proportionally too small to allow the formation of liposomes. The backbone is preferably glycerol or a sugar. The polymer is preferably PEG.

In another aspect the invention is a method treating a mammal with a lipophilic drug comprising combining the drug in an aqueous solution with one or more solubility enhancers including one or more diacyl lipid-polymer conjugates, said diacyl lipid-polymer conjugates represented by the formula

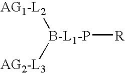

where AG1 and AG2 are acyl groups; where L1, L2 and L3 are linkers; where B is a backbone; where P is a hydrophilic polymer; where R has a molecular weight less than about 200; where said diacyl lipid-polymer conjugate has packing parameters which do not allow for the formation of liposomes; and where said conjugates comprises at least about 20 percent of the combined mass of the drug and all solubility enhancers. The melting temperature of the conjugate is preferably less than about 36 degrees centigrade, and more preferably less than about 25 degrees centigrade. In one embodiment, the head group is proportionally too large to allow the formation of liposomes. In another embodiment, the head group is proportionally too small to allow the formation of liposomes. The backbone is preferably glycerol or a sugar. The polymer is preferably PEG.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

### EXAMPLES

### Example 1

### Increased Solubility of Drug Compounds with PEG-Lipid Conjugates Having Head Groups Too Large for Liposome Formation (Predictive Example)

Various hydrophobic drug compounds are tested for increased aqueous solubility in the presence of compounds listed in Table 6. Conjugates in the table are represented by Chemical structure 2.

TABLE 6
Polymer-lipid conjugates with relatively large head groups

| | | | | Melting |
|---|---|---|---|---|
| # | B | AG1/AG2 | Polymer-(R) | temp. |
| 1 | 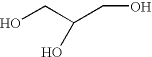 Glycerol | dimyristate | PEG-23-(methyl) | Fluid at 25° C. |
| 2 | 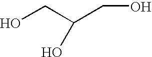 Glycerol | diolealate | PEG-23-(methyl) | Fluid at 25° C. |
| 3 | 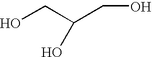 Glycerol | dilaurate | PEG-23-(methyl) | Fluid at 25° C. |
| 4 | 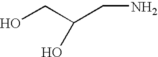 3-aminopropane-1,2-diol | diolealate | PEG-30-(methyl) | Fluid at 45° C. |
| 5 | 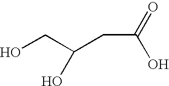 3,4-dihydroxybutanoic acid | diolealate | PEG-23-(1-ornithine) | Fluid at 37° C. |
| 6 | 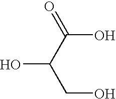 glyceric acid | dimyristate | PEG-23-(methyl) | Fluid at 37° C. |
| 7 | 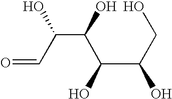 Glucose | dilaurate | PEG-23-(methyl) | Fluid at 37° C. |
| 8 | 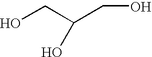 Glycerol | diolealate | PEG-23-(sucrose) | Fluid at 37° C. |
| 9 | 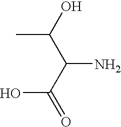 Threonine | dimyristate | PEG-23-(methyl) | Fluid at 37° C. |
| 10 | 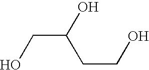 1,2,4-Butanediol | diolealate | PEG-23-(methyl) | Fluid at 37° C. |
| 11 | 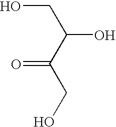 Erythrulose | dilaurate | PEG-23-(methyl) | Fluid at 37° C. |
| 12 | 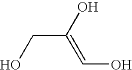 (Z)-prop-1-ene-1,2,3-triol | dimyristate | PEG-23-(methyl) | Fluid at 37° C. |
| 13 | 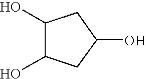 cyclopentane-1,2,4-triol | dilaurate | PEG-23-(methyl) | Fluid at 37° C. |

Compounds 1-3 from table 6 also appear in Table 1 as PEG-23 GDM, PEG-23 GDO and PEG-23 GDL, respectively. The acyl groups and hydrophilic polymers of the other compounds are linked to the backbone by using any type of linker listed. It is favorable to keep AG1 and AG2 close to each other regardless of the type of backbone.

The conjugates are liquid at room temperature or, alternately, are melted before premixing with each drug compound. For example, 40 mg of a selected drug is first premixed with the polymer-lipid conjugate at a w/w ratio of 1 to 5. An aqueous solution of 20 mM phosphate buffer (pH 6) is added with further mixing to result in a total volume of 2 mL. An aliquot of the solution is then tested for drug concentration. In every case, the presence of the conjugate increases the concentration of drug in solution above the concentration of the drug in solution without the conjugate.

The compounds tested are amphotericin B, itraconazole, posaconazole, cyclosporine, and indomethacin.

The physical form of the aqueous solutions of polymer-lipid conjugates is examined, both with and without the presence of the drug compound. Less than 10 percent of the conjugate or conjugate/drug complexes are in the form of liposomes.

### Example 2

### Increased Solubility of Drug Compounds with PEG-Lipid Conjugates Having Head Groups Too Small for Liposome Formation (Predictive Example)

Various hydrophobic drug compounds are tested for increased aqueous solubility in the presence of compounds listed in Table 7. Conjugates in the table are represented by Chemical structure 2.

TABLE 7
Polymer-lipid conjugates with relatively small head groups

| # | B | AG1/AG2 | Polymer —(R) | Melting temp. |
|---|---|---|---|---|
| 1 | 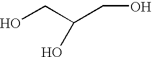 Glycerol | Arachidonic acid/ Arachidonic acid | PEG-5-(methyl) | Fluid at 25° C. |
| 2 | 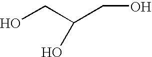 Glycerol | Linoleic acid/ Linoleic acid | Poly-L-lysine 500- (methyl) | Fluid at 25° C. |
| 3 | 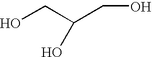 Glycerol | dimyristate | Hexaethylene glycol- (methyl) | Fluid at 25° C. |
| 4 | 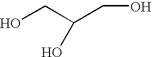 Glycerol | diolealate | Pentaethylene glycol- (methyl) | Fluid at 25° C. |
| 5 | 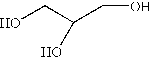 Glycerol | dilaurate | Hexaethylene Glycol- (methyl) | Fluid at 25° C. |
| 6 | 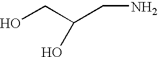 2-aminopropane-1,2-diol | diolealate | Noneethylene Glycol- (l-ornithine) | Fluid at 25° C. |
| 7 | 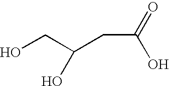 3,4-dihydrobutanoic acid | diolealate | Octaethylene Glycol- (l-ornithine) | Fluid at 25° C. |
| 8 | 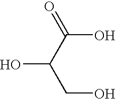 glyceric acid | dimyristate | Hexaethylene glycol- (methyl) | Fluid at 25° C. |
| 9 | 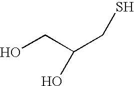 3-mercaptopropane-1,2-diol | diolealate | Pentaethylene glycol- (methyl) | Fluid at 25° C. |
| 10 | 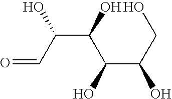 Glucose | dilaurate | Heptaethylene glycol- (methyl) | Fluid at 25° C. |
| 11 | 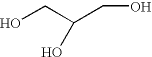 Glycerol | diolealate | Hexaethylene glycol- (sucrose) | Fluid at 25° C. |
| 12 | 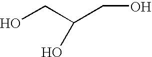 Glycerol | diolealate | Octaethylene glycol- (sucrose) | Fluid at 25° C. |
| 13 | 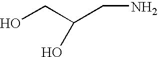 3-aminopropane-1,2-diol | diolealate | Poly(dimer acid-co- erthylene glycol) Mw 2000 | Fluid at 25° C. |
| 14 | 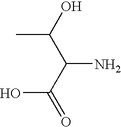 Threonine | dimyristate | Octaethylene glycol- (methyl) | Fluid at 25° C. |
| 15 | 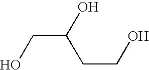 1,2,4-Butanetriol | diolealate | Hexaethylene glycol- (methyl) | Fluid at 25° C. |
| 16 | 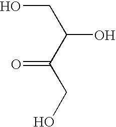 Erythulose | dilaurate | Octaethylene glycol- (methyl) | Fluid at 25° C. |
| 17 | 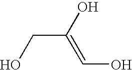 (Z)-prop-1-ene-1,2,3-triol | dimyristate | Hexaethylene glycol- (methyl) | Fluid at 25° C. |
| 18 | 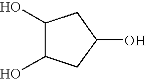 cyclopentane-1,2,4-triol | dilaurate | Pentaethylene glycol- (methyl) | Fluid at 25° C. |

The acyl groups and hydrophilic polymers may be linked to the backbone by using any type of linker listed. However, because it is critical to limit the size of the headgroup in these embodiments, linkers should be selected so as to keep the total molecular weight of the extended backbone to less than about 250. It is favorable to keep AG1 and AG2 close to each other regardless of the type of backbone.

The conjugates are liquid at room temperature or, alternately, are melted before premixing with each drug compound. For example, 40 mg of a selected drug is first premixed with the polymer-lipid conjugate at a w/w ratio of 1 to 8. An aqueous solution of 20 mM phosphate buffer (pH 6) is added with further mixing to result in a total volume of 2 mL. An aliquot of the solution is then tested for drug concentration. In every case, the presence of the conjugate increases the concentration of drug in solution above the concentration of the drug in solution without the conjugate.

The compounds tested are amphotericin B, itraconazole, posaconazole, cyclosporine, and indomethacin.

The physical form of the aqueous solutions of polymer-lipid conjugates is examined, both with and without the presence of the drug compound. Less than 10 percent of the conjugate or conjugate/drug complexes are in the form of liposomes.

### Example 3

### Anticancer Drug Oral Solution

Polymer-lipid conjugate solutions suitable for oral delivery of Gefitinib were prepared. Polymer-lipid was added to a vessel equipped with a mixer propeller. The drug substance was added with constant mixing. Mixing continued until the drug was visually dispersed in the lipids. Pre-dissolved excipients in water were slowly added to the vessel with adequate mixing. Mixing continued until fully a homogenous solution was achieved. A sample formulation is described in Table 8.

TABLE 8

| Ingredient | mg/mL |
|---|---|
| Gefitinib | 5.0 |
| Lipid-polymer Conjugate | 30 |
| Organic Acid | 10 |
| Monosodium phosphate, monohydrate | 1.21 |
| Disodium phosphate, heptahydrate | 0.322 |
| Sodium Hydroxide | See below |
| Hydrochloric Acid | See below |
| Sodium Benzoate | 2.0 |
| Artificial Flavor | 5.0 |
| Purified Water | qs 1 mL |

The PEG-lipid conjugates may be DDGB or any conjugate listed in Table 6 or 7. Sodium hydroxide is used to prepare a 10% w/w solution in purified water. The targeted pH is in a range of 4.0 to 7.0. The NaOH solution is used to adjust pH if necessary. The drug to lipid (i.e., solubility enhancer) ratio is preferably greater than about 1 to 20, and more preferably greater than about 1 to 5. The organic acid may be lactic acid or pyruvic acid or glycolic acid, though lactic acid is most preferable. The concentration of organic acid is preferably in the range 1 and 10%, and more preferably about 2 to 5%.

### Example 4

### Anticancer Drug IV Injectable Solution

The IV solution was prepared as in Example 3, except that no artificial flavor or preservatives added and the targeted pH range was between 6.5 and 7.5. A sample formulation is described in Table 9.

TABLE 9

| Ingredient | mg/mL |
|---|---|
| Gefitinib | 5.0 |
| Lipid-polymer Conjugate | 30 |
| Organic Acid | 10 |
| Monosodium phosphate, monohydrate | 1.21 |
| Disodium phosphate, heptahydrate | 0.322 |
| Sodium Hydroxide | See below |
| Hydrochloric Acid | See below |
| Purified Water | qs 1 mL |

The PEG-lipid conjugates may be DDGB or any conjugates listed in Table 6 or 7. Acyl groups are attached to the glycerol backbone via carboxyl linkages. Sodium hydroxide is used to prepare a 10% w/w solution in purified water. The targeted pH is in a range of 4.0 to 7.0. The NaOH solution is used to adjust pH if necessary. The drug to lipid ratio is preferably greater than about 1 to 20, and more preferably greater than about 1 to 5. The organic acid may be lactic acid or pyruvic acid or glycolic acid, though lactic acid is most preferable. The concentration of organic acid is preferably in the range 1 and 5%, and more preferably about 1 to 3%.

### Example 5

### Antifungal Topical Cream

A PEG lipid conjugate was added to a stainless steel vessel equipped with propeller type mixing blades. The drug substance was added with constant mixing. Mixing continued until the drug was visually dispersed in the lipids at a temperature to 60°-65° C. Organic acid, cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 9.

TABLE 10

| Ingredient | % |
|---|---|
| Drug Substance | 1.0 |
| Lipid-polymer Conjugate | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 5 |
| Sodium hydroxide | See below |
| Purified water | qs 100 |

The drug can be either a fungicide or bactericide including posaconazole, equaconazole. itraconazole, terbinafine and metronidazole The conjugate may be any listed in Table 6 or 7. Organic acid may be lactic acid or pyruvic acid or glycolic acid. Sodium hydroxide is used to adjust pH if necessary. The targeted pH range was between 3.5 and 7.0.

### Example 7

### Antifungal Topical Solution

The topical solution is prepared as in Example 6, except that active was first dissolved in organic acid and ethanol. A sample formulation is described in Table 11.

TABLE 11

| Ingredient | % |
|---|---|
| Drug Substance | 1.0 |
| Lipid-polymer Conjugate | 5.0 |
| α-Tocopherol | 0.5 |
| Organic acid | 2.5 |
| Ethanol | 5.0 |
| Sodium Benzoate | 0.2 |
| Sodium Hydroxide | See Below |
| Purified Water | qs 100 |

The conjugate may be any listed in Table 6 or 7. Organic acid may be lactic acid or pyruvic acid or glycolic acid. Sodium hydroxide is used to adjust pH if necessary. The targeted pH range is between 3.5 and 7.0.

### Example 8

### Synthesis 3,4-Di(oleoyloxy)-decaethylene glycol-benzoate (DDGB)

A solution of 0.10 moles) of 3,4-dihydroxybenzoic acid and 0.1 mole of anhydrous pyridine in 250 mL of benzene was added slowly and with stirring, and under anhydrous conditions, to an ice-cold solution of 0.22 moles of freshly distilled oleoyl chloride in 250 mL of benzene, and the mixture was kept under anhydrous conditions for 20 hours at 40° C. After cooling, 450 mL of chloroform was added to the mixture, which was then filtered. Solvent is evaporated under vacuo to leave a white crude solid which can be further purified as follows: The crude surfactant is purified by chromatography on a silica gel (G60, grade 60, mesh 240-400) column. The column is eluted with a 1:1 (v/v) diethyl ether/acetone mixture. The first fraction eluted is unreacted reagents and discarded. The fraction was collected at a peak of 254 nm (UV monitoring). Dry out of the solvents under vacuo, which yield approximately 70% of 3,4-di(oleoyloxy) benzoic acid.

A mixture of 0.2 mol of 3,4-di(oleyloxy) benzoic acid, 0.2 mol of decaethylene glycol, 0.21 mol of p-toluenesulfonyl chloride, 0.11 mol of 4-dimethylamino pyridine, 0.5 mol of tetrabutylammonium hydrogensulfate, and 0.14 mol of potassium carbonate was suspended in 600 mL of tetrahydrofuran. The reaction mixture was stirred for 22 h at 50° C. The reaction mixture was cooled to room temperature and poured into 1500 mL of water and 300 mL of methylene chloride were added. The solution was then acidified with dilute hydrochloric acid to pH 1. The organic layer was separated, washed five times with 350 mL of water and dried over magnesium sulfate. The solvent was removed under vacuum and the residue was redissolved in 200 mL of ethanol. The colorless solution was kept at 4° C. for 12 hours or until the formation of a white precipitate. Collecting the precipitate by filtration and drying under vacuum yielded approximately 80% of the final product (Chemical Structure 3).

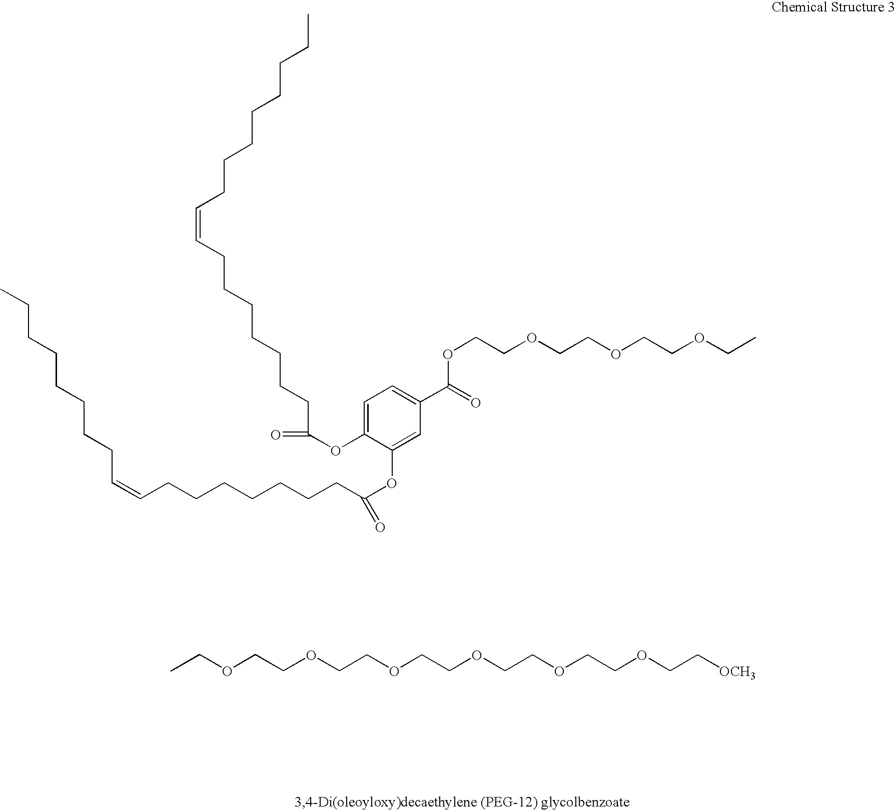

### Example 9

### Pharmacokinetic Profile and Bioavailability of Gefitinib Formulations

Experiments were performed to determine blood levels of Gefitinib formulations after both intravenous and oral dosing. For comparison, Gefitinib formulations were also tested. Groups of three male mice (B6D2F1) were used for the studies. HPLC-MS analyses were performed on heparinized mouse plasma samples obtained typically at 0 hr, 0.08 hr, 0.5 hr, 1 hr, 2 hr, 4 hr, 7 hr, 16 hr and 24 hr after bolus IV injection. After oral feeding, the analyses were performed at 0 hr, 0.5 hr, 1 hr, 2 hr, 4 hr, 8 hr, 16 hr and 24 hr. To determine the level of each drug, the drug was first isolated from plasma with a sample pre-treatment. Acetonitrile were used to remove proteins in samples. An isocratic HPLC-MS/MS method was then used to separate the drugs from any potential interference. Drug levels were measured by MS detection with a multiple reaction monitoring (MRM) mode. PK data was analyzed using the WinNonlin program (ver. 5.2, Pharsight) compartmental models of analysis. The results demonstrated that formulations of compounds in the present invention have a superior PK profile than in Cremophor EL®.

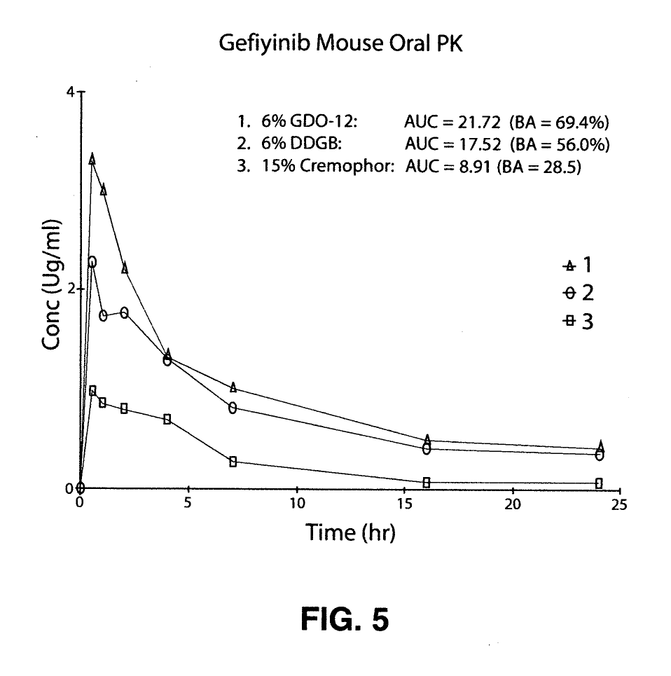

FIG. 5 shows Gefitinib mouse oral PK profiles of (1) 6% PEG-12 GDO in 10 mM sodium phosphate buffer (pH 6.0), (2) 6% DDGB in 10 mM sodium phosphate buffer (pH 6.0) and (3) 15% Cremophor EL® in 10 mM sodium phosphate buffer (pH 6.0). The drug was administered orally and the dosing strength was 25 mg/kg. The drug was also administered intravenously and the dosing strength was 25 mg/kg in 15% Cremophor EL® and in 10 mM sodium phosphate buffer (pH 6.0). The AUC (31.3) obtained from the IV administration was used for calculating the relative bioavailability (BA). The bioavailabilities were 69.4%, 56.0% and 28.5% for GDO-12, DDGB and Cremophor EL®, respectively.

### Example 10

### Solid Dose Formulations

The conjugates of the present invention are useful in preparing solid oral dose formulations of water insoluble drugs. In this aspect, the conjugate or mixture of conjugates are mixed with the drug substance. Then, an excipient having a higher melting point is introduced. The excipient may be melted or dissolved in a suitable solvent. The final mixture is cooled, any solvents are removed, and the mixture is formed into capsules, tablets, or the like.

To prepare a solid dose formulation, a conjugate (about 42% of total mass) is added to a stainless steel vessel equipped with mixing blades. The drug substance (about 15% of total mass) is added with constant mixing. Mixing with heating continues until the drug is virtually dispersed. In a separate container, d-alpha-tocopheryl propylene glycol-1000 succinate (TPGS-VE) or a similar compound (about 42% of total mass) is dissolved in ethanol (about 1% of total mass) and then added to the vessel. Mixing continues until a homogenous solution is achieved. The ethanol is removed by vacuum.

We claim:

1. A composition for enhancing the solubility of a lipophilic drug, said composition comprising at least 25 percent by weight of a diacyl lipid-polymer conjugate, said diacyl lipid-polymer conjugate represented by the formula 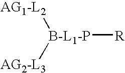
where AG1 and AG2 are acyl groups;
where L1, L2 and L3 are linkers;
where B is a backbone;
where P is polyethyleneglycol;
where R is not cationic;
where AG1 and AG2 together comprise the lipophilic tail portion of the conjugate;
where B, L1, L2, L3, P and R together comprise the hydrophilic head portion of the conjugate; and
where the head portion is too large in relation to the tail portion to allow a pure aqueous solution of the conjugate to predominantly form liposomes.

2. The composition of claim 1, where the melting temperature of the conjugate is less than about 36 degrees centigrade.

3. The composition of claim 1, where the melting temperature of the conjugate is less than about 25 degrees centigrade.

4. The composition of claim 1, where the backbone is glycerol.

5. The composition of claim 1, where the backbone is a sugar.

6. A pharmaceutical composition comprising a lipophilic active agent and one or more solubility enhancers including a diacyl lipid-polymer conjugate, said diacyl lipid-polymer conjugate represented by the formula 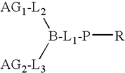
where AG1 and AG2 are acyl groups;
where L1, L2 and L3 are linkers;
where B is a backbone;
where P is polyethyleneglycol;
where R is not cationic;
where AG1 and AG2 together comprise the lipophilic tail portion of the conjugate;
where B, L1, L2, L3, P and R together comprise the hydrophilic head portion of the conjugate; and
where the head portion is too large in relation to the tail portion to allow a pure aqueous solution of the conjugate to predominantly form liposomes; and
where said conjugate comprises at least about 20 percent of the combined mass of the drug and all solubility enhancers.

7. The composition of claim 6, where the melting temperature of the conjugate is less than about 36 degrees centigrade.

8. The composition of claim 6, where the melting temperature of the conjugate is less than about 25 degrees centigrade.

9. The composition of claim 6, where the backbone is glycerol.

10. The composition of claim 6, where the backbone is a sugar.

11. A method of enhancing the solubility of a lipophilic drug comprising
combining the drug in an aqueous solution with one or more solubility enhancers including a diacyl lipid-polymer conjugate, said diacyl lipid-polymer conjugate represented by the formula 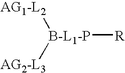
where AG1 and AG2 are acyl groups;
where L1, L2 and L3 are linkers;
where B is a backbone;
where P is polyethyleneglycol;
where R is not cationic;
where AG1 and AG2 together comprise the lipophilic tail portion of the conjugate;
where B, L1, L2, L3, P and R together comprise the hydrophilic head portion of the conjugate;
where the head portion is too large in relation to the tail portion to allow a pure aqueous solution of the conjugate to predominantly form liposomes; and
where said conjugate comprises at least about 20 percent of the combined mass of the drug and all solubility enhancers.

12. The method of claim 11, where the melting temperature of the conjugate is less than about 36 degrees centigrade.

13. The method of claim 11, where the melting temperature of the conjugate is less than about 25 degrees centigrade.

14. The method of claim 11, where the backbone is glycerol.

15. The method of claim 11, where the backbone is a sugar.

16. A method treating a mammal with a lipophilic drug comprising
combining the drug in an aqueous solution with one or more solubility enhancers including a diacyl lipid-polymer conjugate, said diacyl lipid-polymer conjugate represented by the formula 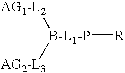
where AG1 and AG2 are acyl groups;
where L1, L2 and L3 are linkers;
where B is a backbone;
where P is polyethyleneglycol;
where R is not cationic;
where AG1 and AG2 together comprise the lipophilic tail portion of the conjugate;
where B, L1, L2, L3, P and R together comprise the hydrophilic head portion of the conjugate;
where the head portion is too large in relation to the tail portion to allow a pure aqueous solution of the conjugate to predominantly form liposomes;
where said conjugate comprises at least about 20 percent of the combined mass of the drug and all solubility enhancers; and
administering the solution to the mammal.

17. The method of claim 16, where the melting temperature of the conjugate is less than about 36 degrees centigrade.

18. The method of claim 16, where the melting temperature of the conjugate is less than about 25 degrees centigrade.

19. The method of claim 16, where the backbone is glycerol.

20. The method of claim 16, where the backbone is a sugar.
